(19) United States
(12) Patent Application Publication
(10) Pub. No.: US 2025/0311944 A1
(43) Pub. Date: Oct. 9, 2025

(54) GAIT BALANCE MONITORING SYSTEM AND GAIT BALANCE MONITORING METHOD

(71) Applicant: National Taipei University, New Taipei City (TW)

(72) Inventors: Bor-Shing LIN, New Taipei City (TW); Huie-You CHEN, Taoyuan City (TW)

(21) Appl. No.: 19/096,645

(22) Filed: Mar. 31, 2025

(30) Foreign Application Priority Data
Apr. 3, 2024 (TW) 113112811

(51) Int. Cl. A61B 5/11; A61B 5/00; G16H 50/30

(52) U.S. Cl. CPC A61B 5/112; A61B 5/1126; A61B 5/7435; A61B 5/7475; G16H 50/30; A61B 2562/0219

(57) ABSTRACT

A gait balance monitoring system includes a sensor and a computing device. The sensor is disposed on torso of a subject. When the subject performs gait tests on a plurality of terrains, the sensor is configured to measure pieces of raw gait data on the plurality of terrains. The computing device is coupled to the sensor, and is configured to perform data processing on the pieces of raw gait data to obtain a plurality of gait data. The computing device is configured to analyze the plurality of gait data to generate a plurality of gait balance score corresponding to the plurality of gait data so as to transmit a plurality of gait data and the plurality of gait balance score to a server.

### CROSS-REFERENCE TO RELATED APPLICATION

This application claims priority to Taiwan Application Serial Number 113112811, filed Apr. 3, 2024, which is herein incorporated by reference in its entirety.

### BACKGROUND

### Field of Invention

The present disclosure relates to an electronic system and a monitoring method. More particularly, the present disclosure relates to a gait balance monitoring system and a monitoring method.

### Description of Related Art

Global aging population has grown rapidly in recent years. Aging trend will be accompanied by an increase in falls caused by poor gait balance, which has become a major issue in the safety of the elderly population. Individual aging causes the balance to decline year by year, thereby increasing the risk of falling.

However, currently, detection of individual balance is still limited to the balance score determined by medical professionals in hospitals or limited by professional-grade equipment in laboratories. With the growth of the elderly population, conventional medical resources are difficult to cope with the need for long-term detection of individual balance.

For the foregoing reasons, there is a need for providing a suitable gait balance monitoring system and a monitoring method to solve the above problems encountered in related art approaches.

### SUMMARY

One aspect of the present disclosure provides a gait balance monitoring system. The gait balance monitoring system includes a sensor and a computing device. The sensor is disposed on a torso of a subject. When the subject performs gait tests on a plurality of terrains, the sensor is configured to measure pieces of raw gait data on the plurality of terrains. The computing device is coupled to the sensor, and is configured to perform data processing on the pieces of raw gait data to obtain a plurality of gait data. The computing device is configured to analyze the plurality of gait data to generate a plurality of gait balance scores corresponding to the plurality of gait data so as to transmit the plurality of gait data and the plurality of gait balance scores to a server.

Another aspect of the present disclosure provides a gait balance monitoring method. The gait balance monitoring method includes following steps: measuring pieces of raw gait data of a subject performing gait tests on a plurality of terrains respectively by a sensor disposed on a torso of the subject; performing data processing on the pieces of raw gait data to obtain a plurality of gait data by a computing device; analyzing the plurality of gait data to generate a plurality of gait balance scores corresponding to the plurality of gait data by the computing device; and transmitting the plurality of gait data and the plurality of gait balance scores to a server by the computing device.

In view of the aforementioned shortcomings and deficiencies of the prior art, the present disclosure provides a technology of a gait balance monitoring system and a gait balance monitoring method. Through the design of gait balance monitoring system and gait balance monitoring method, it is possible to monitor the balance changes of individuals in the long term and allocate medical resources appropriately.

### BRIEF DESCRIPTION OF THE DRAWINGS

The present disclosure can be more fully understood by reading the following detailed description of the embodiment, with reference made to the accompanying drawings as follows:

FIG. 1 depicts a schematic diagram of a gait balance monitoring system and a server according to some embodiments of the present disclosure;

FIG. 2 depicts a schematic diagram of a computing device of a gait balance monitoring system according to some embodiments of the present disclosure;

FIG. 3 depicts a schematic diagram of an user interface of a computing device of a gait balance monitoring system according to some embodiments of the present disclosure;

FIG. 4 depicts a flow chart of a gait balance monitoring method according to some embodiments of the present disclosure;

FIG. 5 depicts a schematic diagram of a terrain task monitored by a gait balance monitoring system according to some embodiments of the present disclosure;

FIG. 6 depicts a schematic diagram of pieces of gait data of a gait balance monitoring system according to some embodiments of the present disclosure;

FIG. 7 depicts a schematic diagram of a terrain task monitored by a gait balance monitoring system according to some embodiments of the present disclosure; and

FIG. 8 depicts a schematic diagram of a terrain task monitored by a gait balance monitoring system according to some embodiments of the present disclosure.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

The terminology used herein is for the purpose of describing particular example embodiments only and is not intended to be limiting of the present disclosure. As used herein, the singular forms “a,” “an” and “the” are intended to include the plural forms as well, unless the context clearly indicates otherwise.

Furthermore, it should be understood that the terms, “comprising”, “including”, “having”, “containing”, “involving” and the like, used herein are open-ended, that is, including but not limited to.

The terms used in this specification and claims, unless otherwise stated, generally have their ordinary meanings in the art, within the context of the disclosure, and in the specific context where each term is used. Certain terms that are used to describe the disclosure are discussed below, or elsewhere in the specification, to provide additional guidance to the practitioner skilled in the art regarding the description of the disclosure.

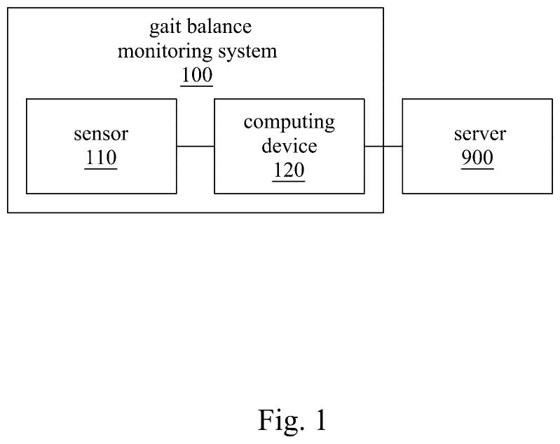

FIG. 1 depicts a schematic diagram of a gait balance monitoring system 100 and a server 900 according to some embodiments of the present disclosure. In one embodiment, the gait balance monitoring system 100 includes a sensor 110 and a computing device 120. The sensor 110 is coupled to the computing device 120. The computing device 120 is coupled to the server 900.

In some embodiments, the sensor 110 is configured to collect pieces of raw gait data of a subject. The sensor 110 includes an inertial measurement unit (IMU), which is configured to nine-axis data. In detail, the sensor 110 can be configured to measure and transmit pieces of data of three-axis acceleration, three-axis angular velocity and three-axis magnetic direction respectively. In some embodiments, the sensor 110 can be implemented as a wearable device so as to be conveniently placed on any part of the subject's body.

In some embodiments, the server 900 is a computer system with powerful computing capabilities. Users can connect to it through their personal mobile devices to request specific information and services.

Due to the decline in fertility and the increase in life expectancy, global aging population has grown rapidly in recent years. Trend of population aging will be accompanied by an increase in falls caused by poor gait balance, which has become a major issue in the safety of the elderly population. According to statistics from the World Health Organization, the prevalence of falls among the elderly has increased year by year in recent years, making falls the second largest cause of accidental injuries. In other words, individual aging causes the balance to decline year by year, thereby increasing the risk of falling.

However, currently, the detection of individual balance is still limited to the balance score determined by professionals in hospitals or limited by professional-grade equipment in laboratories. With the growth of the elderly population, conventional medical resources are difficult to cope with the need for long-term detection of individual balance. The present disclosure will describe how to improve the aforementioned problems in the following paragraphs.

In some embodiments, the computing device 120 is configured to record pieces of raw gait data of the subject, and to run a deep learning model stored in the computing device 120 to analyze and process the pieces of raw gait data to obtain a plurality of gait data of the subject. The computing device 120 is designed to be portable for the subject to monitor the subject's gait over a long period of time. Then, the computing device 120 of the present invention acts as an edge computing device to preliminarily process the pieces of raw gait data raw gait data when close to the subject, so as to quickly analyze and reduce a time of data transmission, thereby reducing privacy and security issues of the subject.

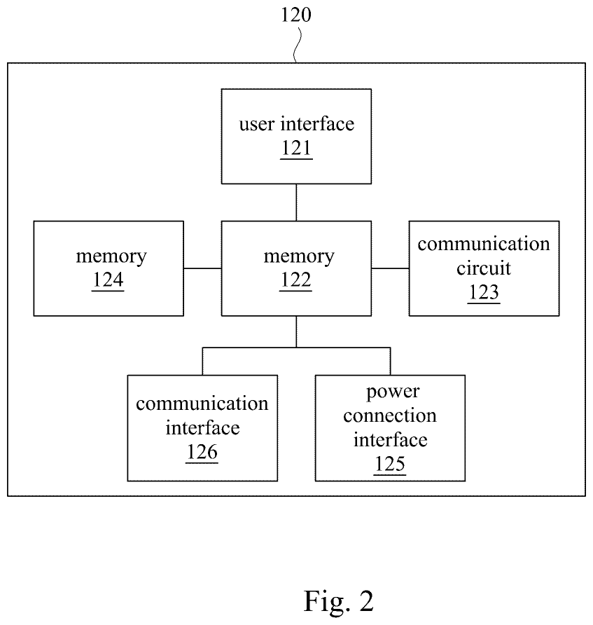

In order to facilitate the understanding internal structure of the computing device 120, please refer to FIG. 2. FIG. 2 depicts a schematic diagram of the computing device 120 of the gait balance monitoring system 100 in FIG. 1 according to some embodiments of the present disclosure. The computing device 120 includes a user interface 121, a processor 122, a communication circuit 123, a memory 124, a power connection interface 125 and a communication interface 126.

In some embodiments, the computing device 120 can be implemented as a field programmable gate array (FPGA) development board. In some embodiments, a size and a shape of the computing device 120 can be designed according to actual needs. The computing device 120 is designed mainly based on the principle of portability. The computing device 120 can be reconfigured and the logic gates on its own development board can be reset. Through the layout planning of an integrated circuit, it can be ensured that the computing resources of the integrated circuit can be flexibly reused.

In some embodiments, the user interface 121 is configured as a medium for a device or a system to interact with the user and exchange information. The user interface 121 of the present disclosure is designed based on characteristics of the elderly population (deterioration of vision, operating ability, hearing, and language expression ability) to provide simple visual patterns and remove lengthy design interfaces, thereby making it easier for the elderly population to use. Details will be introduced in following paragraphs.

In some embodiments, the processor 122 comprise includes but not limited to a single processor and an integration of many micro-processors, for example, a central processing unit (CPU or a graphic processing unit (GPU).

In some embodiments, the communication circuit 123 includes a Wi-Fi module and Bluetooth module (not shown in the figure). The Wi-Fi module complies with the IEEE 802.11 standards and operates in different frequency bands (e.g., 2.4 GHz or 5 GHz) to transmit data. The Bluetooth module complies with relevant communication standards (e.g., protocol specifications after Bluetooth 4.0) to support interconnection of multiple electronic devices and reduce power consumption of electronic devices.

In some embodiments, the memory 124 includes a flash memory, hard disk drive (HDD), a solid state drive (SSD), a dynamic random access memory (DRAM) or a static random access memory (SRAM). The memory 124 is configured to store the pieces of raw gait data collected by the sensor 110.

In some embodiments, the power connection interface 125 is a connection point between the device and peripheral equipment. Through a input/output port, the computing device 120 establishes a communication channel with the peripheral device to obtain power required by the computing device 120 from a power source.

In some embodiments, the communication interface 126 is configured to allow devices or equipment the same communication standards to connect each other. In some embodiments, the communication interface 126 includes high definition multimedia interface (HDMI) and universal serial bus (USB). In some embodiments, the computing device 120 can also obtain power required by the computing device 120 through the communication interface 126.

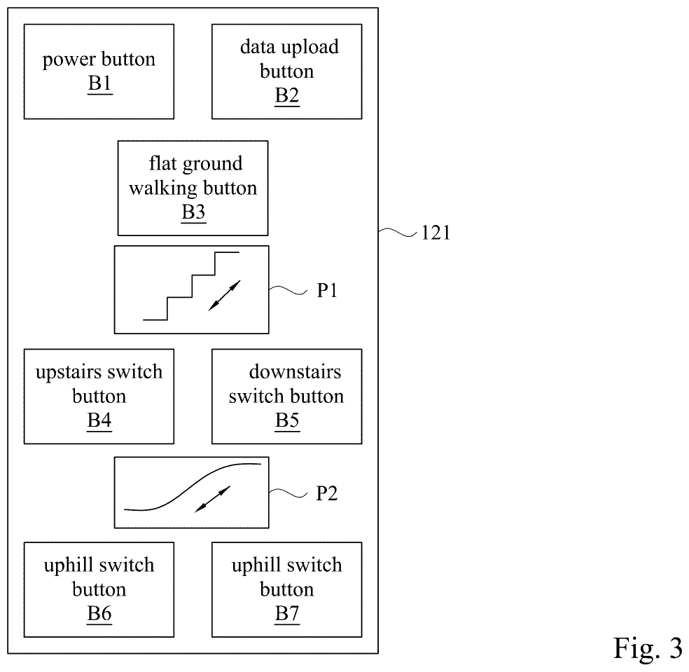

In order to facilitate the understanding design of the user interface 121, please refer to FIG. 1 and FIG. 3. FIG. 3 depicts a schematic diagram of the user interface 121 of the computing device 120 of the gait balance monitoring system 100 according to some embodiments of the present disclosure. The user interface 121 includes a power button B1, a data upload button B2, a plurality of terrain task buttons (e.g., a flat ground walking button B3, a upstairs switch button B4, a downstairs switch button B5, a uphill switch button B6, a downhill switch button B7), a terrain icon P1 and a terrain icon P2.

The power button B1 is configured to turn on and off power of the computing device 120.

The data upload button B2 is configured to allow a user to upload the plurality of gait data processed by the computing device 120 to the server 900, so that the server 900 can further analyze the plurality of gait data and provide sufficient storage space for long-term monitoring and prediction.

The flat ground walking button B3, the upstairs switch button B4, the downstairs switch button B5, the uphill switch button B6 and the downhill switch button B7 correspond to different terrains respectively. Users can switch the flat ground walking button B3, the upstairs switch button B4, the downstairs switch button B5, the uphill switch button B6 and the downhill switch button B7 to allow the computing device 120 to collect and record the pieces of raw gait data of different terrains corresponding to the sensor 110.

The terrain icon P1 corresponds to diagrams of going up and down stairs to provide simple visual patterns, making it convenient for the elderly to operate. The terrain icon P2 corresponds to uphill and downhill diagrams to provide simple visual patterns, making it easier for the elderly population to operate.

It is further explained that the designs of the user interface 121 are only examples to illustrate some possible ways of integrating and separately setting the functional blocks in the foregoing embodiments, and the present disclosure is not limited thereto. It will be understood by those of ordinary skill in the art that various modifications and applications may be made without departing from essential characteristics of the aspects. For example, the elements described in detail in the above aspects may be modified. In addition, differences related to these modifications and applications should be construed as being covered by the scope of the invention as defined by the following claims. In addition, differences between going up and down stairs and going up and down slopes will be explained in following operations.

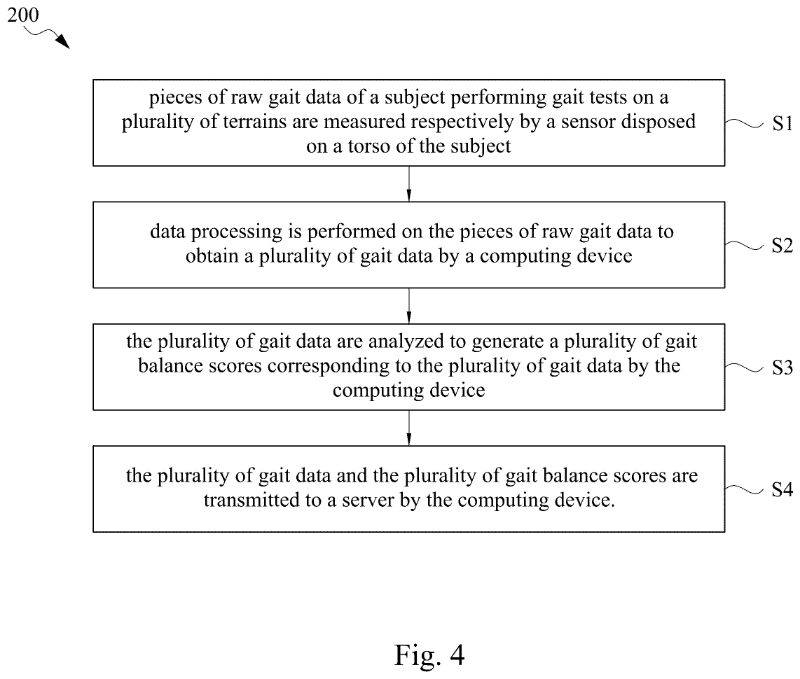

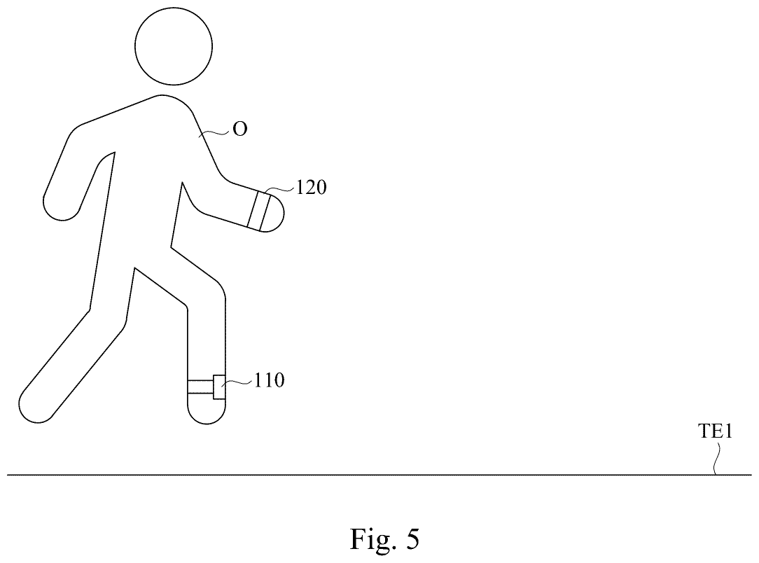

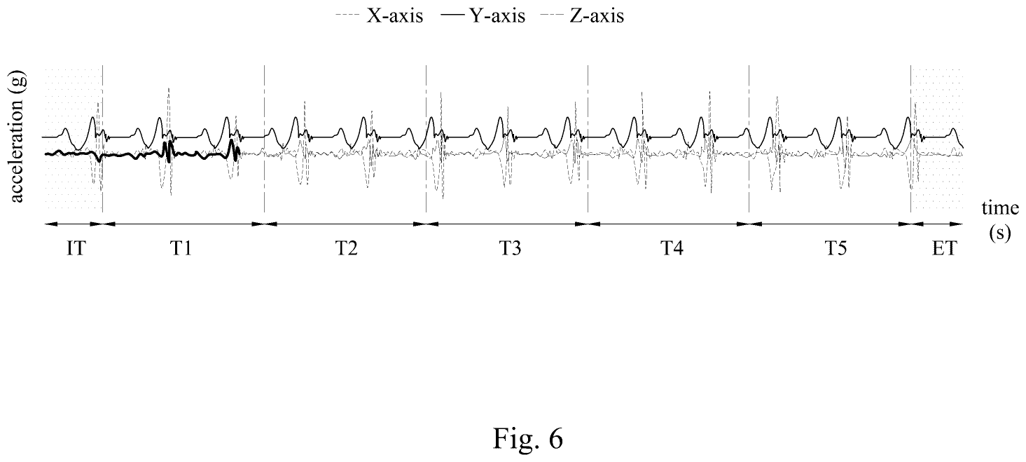

In order to facilitate the understanding operation of the gait balance monitoring system 100 of the present disclosure, please refer to FIG. 3 to FIG. 6. FIG. 4 depicts a flow chart of a gait balance monitoring method 200 according to some embodiments of the present disclosure. FIG. 5 depicts a schematic diagram of a terrain task monitored by a gait balance monitoring system 100 in FIG. 1 according to some embodiments of the present disclosure. FIG. 6 depicts a schematic diagram of pieces of gait data of a gait balance monitoring system 100 in FIG. 1 according to some embodiments of the present disclosure. The gait balance monitoring method 200 includes step S1 to step S4. The gait balance monitoring method 200 can be executed by the gait balance monitoring system 100 in FIG. 1.

In step S1, please refer to FIG. 3 to FIG. 6, the sensor 110 disposed on a lower limb of a subject O is configured to measure pieces of raw gait data (e.g.: three-axis acceleration variation diagram shown in FIG. 6) of the subject O performing a gait test on a terrain TE1 (e.g.: flat ground). It should be noted that a distance for the subject O to perform the gait test must be at least larger than 10 meters and completed once. For example, the subject O walks about 15 meters straight on terrain TE1 (e.g.: flat ground) to complete a test, which is repeated three times, with an interval of about 30 seconds between each test. It is further explained that before the test begins, the subject O presses the flat ground walking button B3 of the user interface 121 of the computing device 120 corresponding to the terrain TE1 (e.g., flat ground) so that the computing device 120 corresponding to the sensor 110 collects the pieces of raw gait data of the terrain TE1 (e.g., flat ground) for recording. Values of the aforementioned embodiments can be designed according to actual needs and are not limited to the embodiments of the present disclosure.

In some embodiments, the sensor 110 can be disposed on the torso of the subject O, for example: left or right ankle, thigh, left and right wrist or back. The computing device 120 can be disposed on the wrist of the subject O or in a pocket of clothing. In some embodiments, the sensor 110 and the computing device 120 can be integrated into the same electronic device. For example, electronic devices such as mobile phones, sports watches/rings, or sports anklets can perform sensing and edge computing functions respectively.

In step S2, please refer to FIG. 4 to FIG. 6, the computing device 120 is configured to processes the pieces of raw gait data (e.g., the pieces of raw gait data corresponding to the terrain TE1) to obtain a plurality of gait data.

The pieces of raw gait data in FIG. 6 is composed of three-axis data (e.g., X-axis acceleration, Y-axis acceleration and Z-axis acceleration in the figure) of a test start stage IT, test stages T1-T5 and a test end stage ET. X-axis and Z-axis are parallel to the ground. The Y-axis is perpendicular to the ground.

Please refer to FIG. 5 and FIG. 6, the pieces of raw gait data at the test start stage IT and the test end stage ET will be affected by the body size difference of the subject O. In detail, there is usually noise in the pieces of raw gait data cause by the gait sway of the subject O at the beginning and end of walking. Therefore, the present disclosure will first eliminate the pieces of raw gait data of the test start stage IT and the test end stage ET, and capture the pieces of raw gait data of the test stage (e.g., test stage T1˜T5) and divide it according to the total test time (e.g., each second is divided into 1 equal part) as a plurality of gait data. It should be noted that the present disclosure appropriately sets time length of each of the test stages T1-T5 to ensure that characteristics of changes in the gait data are not lost.

Then, the computing device 120 is configured to standardize the pieces of raw gait data of the subject O in the test phases T1 to T5 according to a test set (i.e., the average of the pieces of raw gait data of a plurality of different subjects) and a standard deviation of the corresponding test set to eliminate the differences in individual body shapes.

In step S3, the computing device 120 is configured to analyze the plurality of gait data to generate a plurality of gait balance scores corresponding to the plurality of gait data.

In some embodiments, the computing device 120 includes a gait balance score assessment model (not shown in the figured). The gait balance score assessment model is configured to analyze the plurality of gait data to generate the plurality of gait balance score corresponding to the plurality of gait data. In some embodiments, the gait balance score assessment model includes an artificial neural network model. An artificial neural network type of the gait balance score evaluation model includes at least one of a convolutional neural network model (CNN), a long short-term memory model (LSTM) and a gated recurrent unit model (GRU). It is further explained that types of the above neural network models are only examples to illustrate some possible ways of integrating and separately setting the functional blocks in the foregoing embodiments, and the present disclosure is not limited thereto. It will be understood by those of ordinary skill in the art that various modifications and applications may be made without departing from essential characteristics of the aspects. For example, the elements described in detail in the above aspects may be modified. In addition, differences related to these modifications and applications should be construed as being covered by the scope of the invention as defined by the following claims. In addition, differences between going up and down stairs and going up and down slopes will be explained in following operations.

Detail training method of the gait balance score assessment model of the computing device 120 will be described in the following paragraphs. The training method of the gait balance score assessment model of the present disclosure is related to an experiment conducted in many universities and community activity centers in Taiwan. Institutional Review Board (IRB) of Taipei Medical University approved this study. This study adhered to principles of the Declaration of Helsinki and provided written informed consent from the subjects or their guardians.

The experiment required a plurality of subjects to complete 14 movements in the Berg Balance Scale (BBS) and receive balance scores assessed by medical professionals (e.g., physical therapists). The scores ranged from 0 to 56, and the higher the score, the better the subject's balance ability. Then, after evaluation by the medical professionals, the test will require a plurality of subjects to wear the sensor 110 and the computing device 120 of the gait balance monitoring system 100 of the present disclosure, and walk straight for about 15 meters on flat ground to complete a test, and repeat it six times, with an interval of about 30 seconds(s) between each test, so as to collect a plurality of gait-related data corresponding to the plurality of subjects through the gait balance monitoring system 100. It should be noted that the Berg Balance Scale can be replaced by other scales, such as the Timed Up and Go (TUG) test, which only has one test action.

Further, the data processing method of the plurality of gait-related data is similar to the data processing method of the aforementioned raw gait data. The plurality of processed gait-related data will be used as plurality of training gait data. In the present disclosure, the plurality of training gait data and normalized balance scores corresponding to the plurality of training gait data are configured to train the gait balance score assessment model.

In some embodiments, the present disclosure uses the plurality of training gait data as a data set, and uses cross-validation to randomly divide the data in the data set into a test set and a training set, thereby repeatedly training the gait balance score assessment model to learn how to generate a gait balance score. For example, the plurality of training gait data of 120 have been collected, and divided into K equal parts (e.g., 5 equal parts) through K-Fold Cross-Validation method. The training gait data of K−1 equal parts (e.g., 4 equal parts, i.e., 96 subjects) are used as the training set and the training gait data of 1 equal part (i.e., 24 subjects) are used as the test set, and the corresponding balance scores (i.e., the scores evaluated by medical professionals) are paired to train the gait balance score assessment model. Number of equal parts can be designed according to actual needs and is not limited to the embodiment of the present disclosure.

Finally, please refer to FIG. 2 and FIG. 5, the trained gait balance score assessment model is transplanted to the computing device 120 of the gait balance monitoring system 100 in the present disclosure, so that the computing device 120 first performs edge computing on the pieces of raw gait data of different terrains (e.g., flat ground, stairs and slopes) to obtain the gait data, and then generates a gait balance score for the gait data through the gait balance score assessment model of the computing device 120.

In step S4, please refer to FIG. 1, FIG. 3 to FIG. 5, an operation instruction of the subject O is received through the data upload button B2 of the user interface 121 of the computing device 120, so that the computing device 120 is configured to transmit the plurality of gait data and gait balance scores for the terrain TE1 (e.g., flat ground) to the server 900.

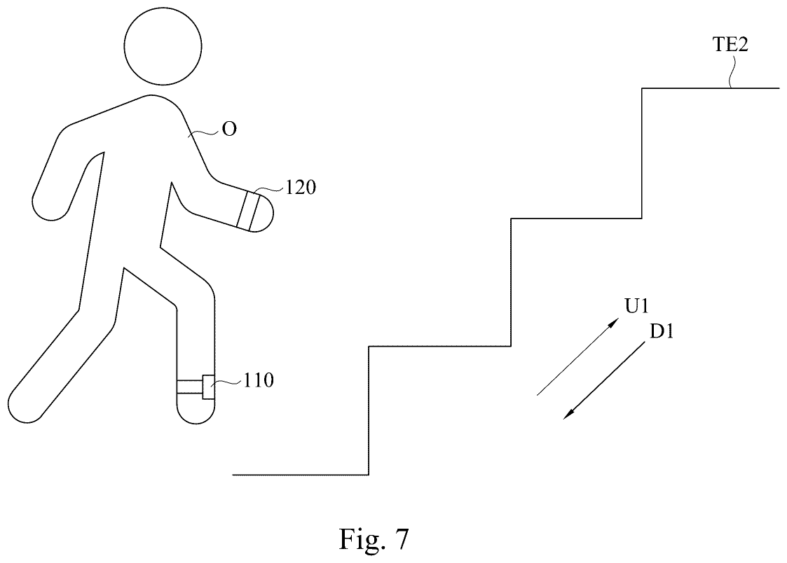

FIG. 7 depicts a schematic diagram of a terrain task monitored by a gait balance monitoring system 100 in FIG. 1 according to some embodiments of the present disclosure. Compared to the embodiment of FIG. 5, there are several differences between the embodiment of FIG. 5 and the embodiment of FIG. 7. The first difference is that the terrain TE1 (e.g., flat ground) on which subject O performed the gait test was changed to terrain TE2 (e.g., stairs). The second difference is that in the gait test, the subject O performed the upstairs test U1 and the downstairs test D1 respectively on terrain TE2 (e.g., stairs). The third difference is that the subject O operates the upstairs switch button B4 and the downstairs switch button B5 of the user interface 121 of FIG. 3, so that the computing device 120 records the pieces of raw gait data corresponding to the terrain TE2 (eg, stairs) collected by the sensor 110. The gait data collection and processing methods of the terrain TE2 are similar to those of the terrain TE1, and detail repetitious descriptions are omitted here.

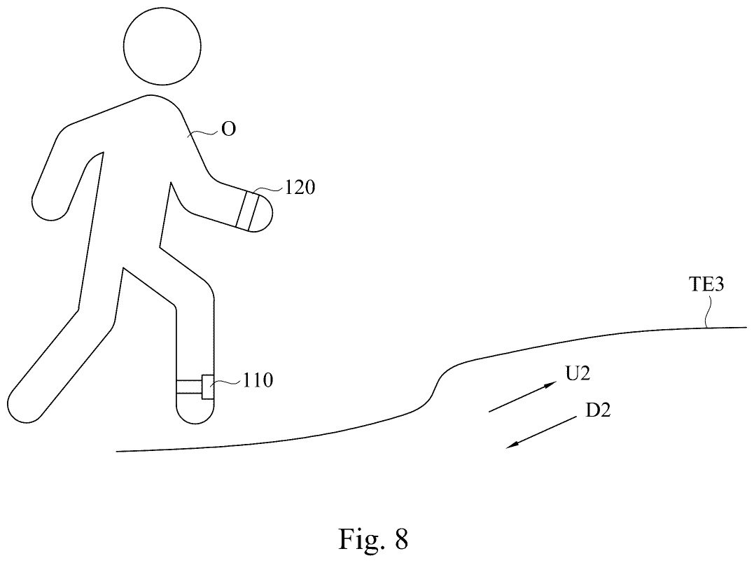

FIG. 8 depicts a schematic diagram of a terrain task monitored by a gait balance monitoring system 100 in FIG. 1 according to some embodiments of the present disclosure. Compared to the embodiment of FIG. 5, there are several differences between the embodiment of FIG. 5 and the embodiment of FIG. 8. The first difference is that the terrain TE1 (e.g., flat ground) on which subject O performed the gait test was changed to terrain TE3 (e.g., a gentle slope). The second difference is that in the gait test, the subject O performed uphill test U2 and downhill test D2 respectively on terrain TE3 (e.g., a gentle slope). The third difference is that the subject O operates the uphill switch button B6 and the downhill switch button B7 of the user interface 121 of FIG. 3 so that the computing device 120 records the pieces of raw gait data corresponding to the terrain TE3 (e.g., a gentle slope) collected by the sensor 110. The gait data collection and processing methods of the terrain TE3 are similar to those of the terrain TE1, and detail repetitious descriptions are omitted here.

It should be noted that since the terrain TE2 (e.g., stairs) is a step-by-step movement, the subject O tends to support himself on one leg during the upstairs test U1 and the downstairs test D1. Therefore, during the upstairs test U1 and the downstairs test D1, the gait balance score of the subject O evaluated by the gait balance monitoring system 100 will be significantly lower than the gait balance score on the terrain TE1.

The terrain TE3 (e.g., a gentle slope) is a continuous movement close to flat ground, but with an angular potential conversion. Therefore, during the uphill test U2 and the downhill test D2, the gait balance score of the subject O evaluated by the gait balance monitoring system 100 is between the gait balance score of the terrain TE1 and the gait balance score of the terrain TE2.

It is further explained that the present disclosure uses a trained neural network model (e.g., a gait balance score assessment model) to quantify the gait balance scores of subjects undergoing gait tests on different terrains (e.g., stairs and gentle slopes) to provide reference for medical personnel.

Based on the aforementioned embodiments, the present disclosure designs of a gait balance monitoring system and a gait balance monitoring method to collect the gait data of the subjects, and uses a device with edge computing function to process the data in advance and generate gait balance scores for different terrains. In addition, the gait data and gait balance scores are uploaded to the cloud server for medical staff to refer to during consultation. Besides, under long-term monitoring, if the subject's gait balance score decreases, medical personnel can be notified to give the subject timely treatment, thereby reducing the chance of falling due to aging and allowing medical resources to be properly allocated.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the present disclosure. In view of the foregoing, it is intended that the present disclosure cover modifications and variations of the present disclosure provided they fall within the scope of the following claims.

What is claimed is:

1. A gait balance monitoring system, comprise:
a sensor, disposed on a torso of a subject, wherein when the subject performs gait tests on a plurality of terrains, the sensor is configured to measure pieces of raw gait data on the plurality of terrains; and
a computing device, coupled to the sensor, and configured to perform data processing on the pieces of raw gait data to obtain a plurality of gait data, wherein the computing device is configured to analyze the plurality of gait data to generate a plurality of gait balance scores corresponding to the plurality of gait data so as to transmit the plurality of gait data and the plurality of gait balance scores to a server.

2. The gait balance monitoring system of claim 1, wherein the plurality of terrains comprise one of a flat ground, a slop and stairs, wherein the computing device comprises an user interface, the user interface comprises a plurality of terrain task buttons, wherein the terrain task buttons are configured to receive an operation instruction of the subject so that the computing device switches to one of a plurality of recording modes corresponding to the terrains, so as to respectively record the pieces of raw gait data corresponding to the plurality of terrains.

3. The gait balance monitoring system of claim 2, wherein the computing device is further configured to capture part of the pieces of raw gait data and segment the pieces of raw gait data to obtain the plurality of gait data.

4. The gait balance monitoring system of claim 1, wherein the computing device comprises:
a gait balance score assessment model, configured to analyze the plurality of gait data to generate the plurality of gait balance score corresponding to the plurality of gait data.

5. The gait balance monitoring system of claim 4, wherein the gait balance score assessment model is further configured to receive a plurality of training gait data and a balance score corresponding to the plurality of training gait data so as to identify the training gait data according to the balance score to generate the gait balance scores according to the plurality of gait data.

6. The gait balance monitoring system of claim 4, wherein the gait balance score assessment model comprises at least one of a convolutional neural network model (CNN), a long short-term memory model (LSTM) and a gated recurrent unit model (GRU).

7. The gait balance monitoring system of claim 1, wherein the computing device comprise:
a memory, configured to store the pieces of raw gait data collected by the sensor.

8. The gait balance monitoring system of claim 1, wherein the sensor comprise an inertial measurement unit (IMU).

9. A gait balance monitoring method, comprising:
measuring pieces of raw gait data of a subject performing gait tests on a plurality of terrains respectively by a sensor disposed on a torso of the subject;
performing data processing on the pieces of raw gait data to obtain a plurality of gait data by a computing device;
analyzing the plurality of gait data to generate a plurality of gait balance scores corresponding to the plurality of gait data by the computing device; and
transmitting the plurality of gait data and the plurality of gait balance scores to a server by the computing device.

10. The gait balance monitoring method of claim 9, wherein performing data processing on the pieces of raw gait data to obtain the plurality of gait data by the computing device comprises:
receiving an operation instruction of the subject so that the computing device switches to one of a plurality of recording modes corresponding to the terrains by a plurality of terrain task buttons of an user interface of the computing device, so as to respectively record the pieces of raw gait data corresponding to the plurality of terrains.

11. The gait balance monitoring method of claim 10, wherein performing data processing on the pieces of raw gait data to obtain the plurality of gait data by the computing device further comprises:
capturing part of the pieces of raw gait data and segmenting the pieces of raw gait data to obtain the plurality of gait data by the computing device.

12. The gait balance monitoring method of claim 9, wherein analyzing the plurality of gait data to generate the plurality of gait balance scores corresponding to the plurality of gait data by the computing device comprises:
analyzing the plurality of gait data to generate the plurality of gait balance score corresponding to the plurality of gait data by a gait balance score assessment model of the computing device.

13. The gait balance monitoring method of claim 12, further comprising:
receiving a plurality of training gait data and a balance score corresponding to the plurality of training gait data so as to identify the training gait data according to the balance score to generate the gait balance scores according to the plurality of gait data by the gait balance score assessment model.

14. The gait balance monitoring method of claim 12, wherein the gait balance score assessment model comprises at least one of a convolutional neural network model (CNN), a long short-term memory model (LSTM) and a gated recurrent unit model (GRU).

15. The gait balance monitoring method of claim 9, further comprising:
storing the pieces of raw gait data collected by the sensor by a memory of the computing device.
